(19) United States
(12) Patent Application Publication
(10) Pub. No.: US 2004/0059599 A1
(43) Pub. Date: Mar. 25, 2004

(54) Patient management system

(76) Inventor: Michael E. McIvor, St. Petersburg, FL (US)

Correspondence Address: LARSON AND LARSON, 11199 69TH STREET NORTH, LARGO, FL, 33773

(21) Appl. No.: 10/254,202

(22) Filed: Sep. 25, 2002

(51) Int. Cl. G06F 17/60; A61B 5/00

(52) U.S. Cl. 705/2; 455/422.1; 600/300

(57) ABSTRACT

The system educates patients, monitors their progress and proactively intervenes with therapies to avoid hospitalization. Patient communication devices (PCD) are used to query patients daily on their health status. A patient uses the PCD to log onto a first database housed on the Internet. The database uses screen prompts to elicit, among other things, patient weight, blood pressure, and heart rate and transmits the patient information via an encrypted signal to a second local database located in the physician's office. This second database houses the patient's identifying information. The physician uses the second database to query the first database and download the daily patient entries. If a patient alert flags a patient record for intervention, the second database applies treatment algorithms to the problem to devise a directive. If the healthcare provider concurs with the directive, the system sends a message to the patient via his PCD. A third database contains information on all patients and is used to educate patients. This database educates the patients to understand and manage their illness.

### BACKGROUND OF THE INVENTION

1. Field of the Invention.

This invention relates to a system for managing the health of a patient. More particularly, it refers to a system for educating patients having chronic illnesses, such as health failure, diabetes mellitus and chronic lung disease, monitoring their progress and proactively intervening with therapies to avoid hospitalizations and increase the overall quality of life for the patient.

2. Description of the Prior Art.

There are many patient management systems in existence as shown by multiple U.S. Patents.

U.S. Pat. No. 5,772,585 describes a patient management system including scheduling appointments, entering and displaying data to a physician, displaying allergy warnings and recording diagnosis.

U.S. Pat. No. 5,911,132 describes a system for diagnosing and treating a patient via electronic data communication. An epidemiological database facility collects epidemiological transaction records from physicians and hospitals and then provides diagnostic information to physicians to help confirm a patient's diagnosis.

U.S. Pat. No. 5,924,074 describes a system capturing patient data at its source at the time of entry using a graphical user interface with a touch screen. Authorized healthcare providers can then access the patient records.

U.S. Pat. No. 5,966,692 describes a system for monitoring a patient's heart. A remote station has a device for generating a patient electrocardiogram and a transmitter. The system has a central station continuously communicating with the transmitter to receive information from the patient. A database communication with the central station stores patient data.

U.S. Pat. No. 6,032,119 describes a system for collecting a patient's health information from health providers and thereafter storing it on a database. The data is processed at the server and displayed in the patient's home using a video monitor connected to a multi-media processor.

U.S. Pat. No. 6,154,727 describes a system for tracking client visits. Authentication is obtained from biometric parameters obtained from the client by phone and comparing those to biometric parameters in a database.

U.S. Pat. No. 6,168,563 describes a system having a computer-executable protocol providing information to the patient that interactively monitors the patient's health conditions by asking questions and receiving answers from the patient. The remotely located patient apparatus sends the patient's health information through a communication network to the healthcare provider's computer, in which case the healthcare provider can evaluate the information and send an updated patient health script program to the patient.

U.S. Pat. No. 6,190,313 describes a system whereby a patient's physiological condition is analyzed at a patient site using SHEWHART's rules of detection. A patient is notified of a change in heath status. The system has a patient operated sensor for taking physiological measurements and converting them to a data record. A means for notifying the patient is included when one or more of four rules of detection are met.

U.S. Pat. No. 6,230,142 describes a system for analyzing clinical pathway data. Patient identification and visit data elements are stored. The system provides for modifying at least one decision data element within the clinical pathway database.

U.S. Pat. No. 6,246,992 describes a system for monitoring a group of patients having chronic disease conditions. The system generates and displays a group overview chart having one data point for each patient. Supervisory health instructions are transmitted to at least one patient in the group.

U.S. Pat. No. 6,256,613 describes a system that supports consultation of a specialist by a treating physician. There is a client computer and a supervisory host computer for storing all pertinent patient data.

U.S. Pat. No. 6,278,999 describes a system having a centralized database collecting data from a large number of patients. The patient data is used to perform statistical analysis per patient. The data is available to healthcare providers as well as the patient. Privacy is protected by prohibiting access to the individual data except by those specifically authorized by the patient.

U.S. Pat. No. 6,290,646 describes a system having a device interacting with a patient via a modem. The system queries the patient and transmits measured parameters to a computer at a remote site. The measured parameters are then analyzed by the caregiver at the remote site. If tallied scores exceed a predetermined value, a caregiver is notified that the patient requires immediate attention.

U.S. Pat. No. 6,292,783 describes a home health documentation system having a computer for storing medical-related task information with a phone interface to input or retrieve the information. Verification calenders indicate dates associated with medical related tasks.

Many of these systems have the disadvantage of distancing the physician from the patient rather than supplementing their relationship. An improved system is needed wherein two-way interactive messaging between the patient and the physician for convenient daily monitoring with daily intervention targeted to those patients who need intervention on a given day is provided. The improved system should allow for the simultaneous intensification of doctor monitored therapy and increasing patient compliance. In addition, the improved system has to be cost effective thereby enabling the proper care to be more effectively delivered at a high level, but through daily intervention by a physician or physician extender, as necessary.

### SUMMARY OF THE INVENTION

The health care patient management system of this invention educates patients, monitors their progress and proactively intervenes with therapies in response to any decrease in their health progress thereby avoiding unnecessary hospitalization. The system can be used with patients having a multitude of varying conditions, including, but not limited to, heart failure, diabetes mellitus, asthma, infectious diseases and immune deficiencies. The improved system employs a patient communication device (PCD) such as, for example, a cell phone, telephone, personal computer or a personal digital assistant to query patients daily on their health status. A patient uses the PCD to log onto a first database located on the global computer information network, also known as the Internet, and store information regarding their health. Each patient is assigned a unique patient identifier which is transmitted anonymously from the PCD to the first database. A second, local database, located in a physician's office, houses the patient's unique identifier and their respective health condition information. The physician uses the second database to query the first Internet database providing the physician the ability to download daily patient entries. If a patient alert, previously defined in the system, flags a patient record for intervention, the second database applies a predefined treatment algorithm to the problem (collected health data) to devise a proposed treatment intervention. If the healthcare provider (physician or physician extender) concurs with the proposed treatment intervention, the system sends a message to the patient via the patent's PCD. All of this transmitted information is encrypted as an anonymous string of numbers thereby prohibiting the system from being compromised and being linked back to a particular patient. Thus, the patient-doctor confidentiality relationship is maintained at all times.

A third database contains information on all patients. This information is maintained confidentially and is used for system management by the healthcare provider personnel.

### BRIEF DESCRIPTION OF THE DRAWINGS

The invention can be best understood by those having ordinary skill in the art by reference to the following detailed description when considered in conjunction with the accompanying drawings in which:

FIG. 1 is a graphical representation of a Patient Management System of the present invention depicting how a plurality of doctors can be in communication with a patient having a patient communication device;

FIG. 2 is a graphical representation of the Patient Management System of the present invention depicting how a plurality of patients each having their own patient communication device can be in communication with a single doctor;

FIG. 3A is a first of two parts of a flow diagram illustrating the steps that a patent and doctor can take to communicate between one another to provide health status information to the doctor who can then analyze such information and respond accordingly;

FIG. 3B is a second of two parts of the flow diagram illustrating the steps that a patient and doctor can take to communicate between one another to provide health status information to the doctor who can then analyze such information and respond accordingly;

FIG. 4 is a front view of a patient communication device used by the patient in the present invention illustrating one example of a plurality of messages that the patient may have to answer through the input of information on a touch sensitive keypad;

FIG. 5 is a front view of a patient communication device used by the patient in the present invention illustrating another example of a plurality of messages that the patient may have to answer through the input of information on a touch sensitive keypad wherein a range of choices are provided to the patient;

FIG. 6 is a front view of a patient communication device used by the patient in the present invention illustrating yet another example of a plurality of messages that the patient may have to answer through the input of information on a touch sensitive keypad wherein a “yes” or “no” choice is requested;

FIG. 7 is a front view of a patient communication device used by the patient in the present invention illustrating yet still another example of a plurality of messages that the patient may have to answer through acknowledgment of an instruction sent by a doctor or doctor's office on a touch sensitive keypad; and

FIG. 8 is a front view of a Personal Computer used by a doctor in the present invention illustrating one example of a plurality of decisions that the doctor may make in administrating health care to a patient in communication with the doctor through the use of the patient communication device.

### DETAILED DESCRIPTION OF THE PREFERRED EMBODIMENT

Throughout the following detailed description, the same reference numerals refer to the same elements in all figures.

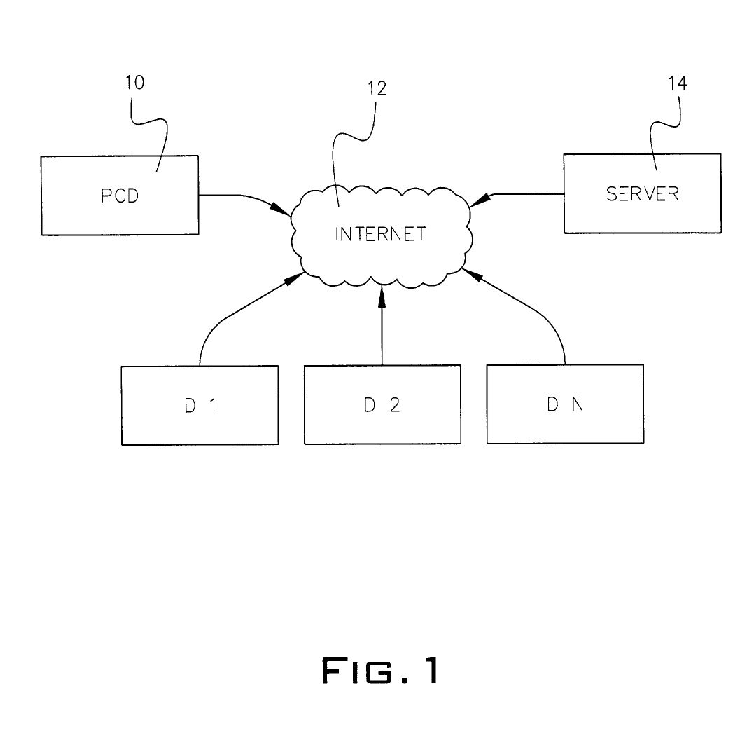

Referring to FIG. 1, the novel patient management system of the present invention is shown wherein a patient, under the care of a healthcare provider (physician), possesses a patient communication device (PCD) 10 which is in communication with a global computer information network, such as the Internet 12. In the preferred embodiment, PCD 10 operates under a wireless access protocol (WAP), such as cellular phone technology. However, nothing herein limits that PCD 10 operate under any other known communication or access protocol, either wireless or hardwired. For instance, HTML (hyper-text marked-up language), XML or other known operating systems could be used. A first intelligent database, or Server 14, is coupled and therefore in communication with the Internet 12. In the preferred embodiment, Server 14 is connected to the Internet 12 by means of a hardwired connection. However, nothing herein limits the use of a wireless connection for Server 14. Since both PCD 10 and Server 14 are in communication with the Internet 12, it is possible for PCD 10 to establish a communication link with Server 14. An alternate embodiment permits the patient to use a personal computer to directly log onto the Server 14 through the Internet by use of a web page accessible through the corporate database. This of course would alleviate the need for PCD 10.

With continuing reference to FIG. 1, it is shown that a healthcare provider, or Doctor Dl, can also be in communication with the Internet 12 and in communication with the patient in possession of PCD 10. In fact, a plurality of healthcare providers, or D1, D2 through DN (wherein N represents an infinite number), can be in communication with the Internet 12 and in direct communication with the single patient. In the example illustrated in FIG. 1, the single patient having PCD 10 in her possession can be in communication with a plurality of healthcare providers, such as, for example, her cardiologist, her hematologist and her family doctor. Accordingly, PCD 10 assigned to one patient can be used to communicate with a plurality of doctors. The communication protocols used by the healthcare providers can be hardwired (land lines) or wireless. In the preferred embodiment, a hardwired communication protocol is used, such as, for example, a cable modem, a DSL line or dial up connection communicating through the Internet. Although not shown in FIG. 1, a second intelligent database is located within the office of each healthcare provider, D1-DN, which are each used in establishing a communication link between Server 14 through the Internet 12 or to PCD 10 directly.

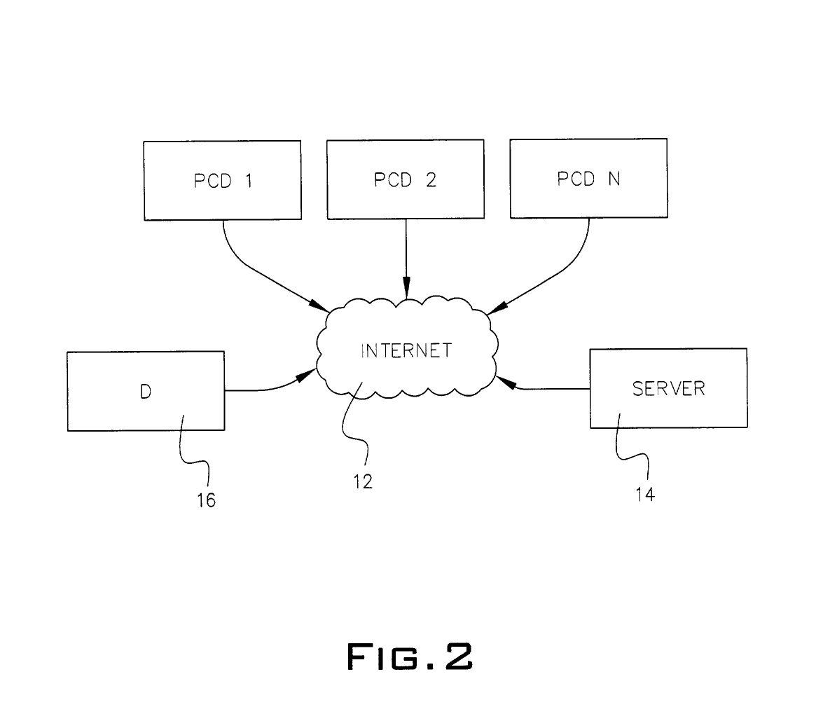

Referring to FIG. 2, it is illustrated that a single healthcare provider, or Doctor 16, can use the patient management system of the present invention to communicate with a plurality of patients, PCD1, PCD2 an PCDN (wherein N represents an infinite number of patients). As stated before, Doctor 16 has a second intelligent database (although not shown in FIG. 2) located in or in close proximity to the office of Doctor 16. FIG. 2. illustrates the preferred embodiment wherein a single Doctor 16, such as, for example, a cardiologist, keeps track of all of the patients under his care that are suffering from heart failure or other heart disease.

Each PCD, of both FIGS. 1 and 2, have a unique patient identifier, or ID number, containing a host of information, including, but not limited to, a patient ID, a physician ID, state of origin and relevant Medicare, Medicaid or insurance company information. When this number is transmitted during a communication establishment, it is encrypted as an anonymous string of numbers so that patient confidentiality and HIPPA compliance can be maintained.

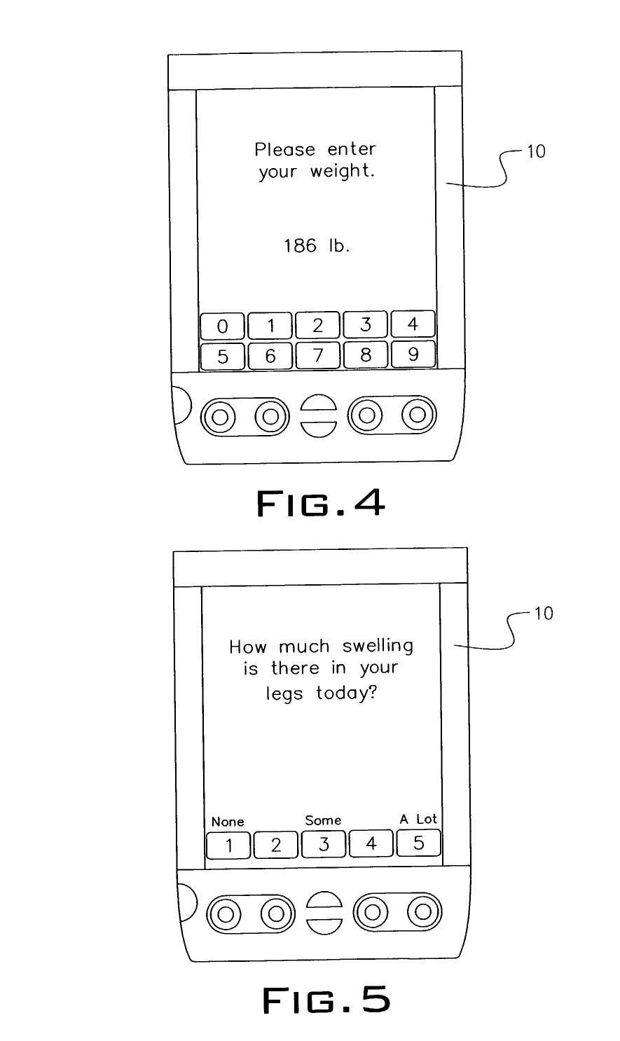

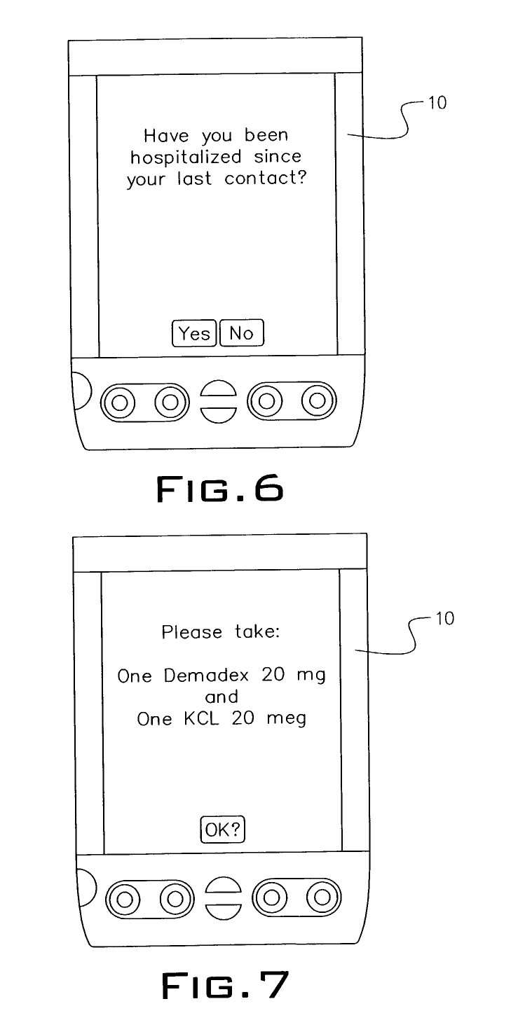

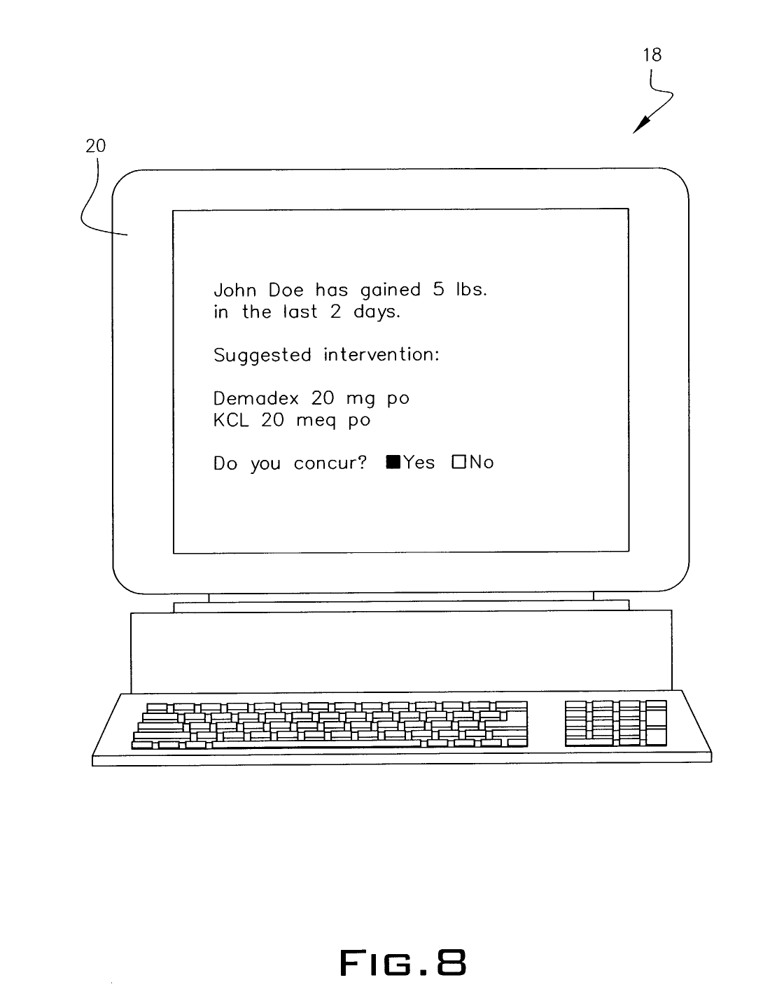

Referring to FIGS. 4 through 7, a PCD 10 is shown wherein it is illustrated that a series of questions and directives are provided to the patient in control of PCD 10. These questions and directives are numerous and can vary depending on the illness and its severity. Accordingly, FIGS. 4 through 7 are merely meant to be illustrative and do not represent the totality of questions and directives that can be transmitted to PCD 10. However, as shown in FIG. 4, PCD 10 can be posed with a question, once a communication link has been established with Server 14, that asks the patient to enter his or her weight. As shown in FIG. 5, a question is being asked of the patient whether a certain condition may be occurring (in this example swelling of the legs) which may be indicative of a problem or condition of the sickness or disease of the patient which may require intervention. FIGS. 4 and 5 illustrate questions which may cause an alert at the doctor's office once the information has been received by the second intelligent database at the doctor's office. FIG. 6 illustrates a question which is asked to determine whether the patient has sought any healthcare from any other providers (i.e., from a hospital) since the last contact with the doctor in communication with the patient in possession of PCD 10. This question may also trigger an alert at the doctor's office, since the doctor may not want to intervene with a specific directive if the patient has already received care relating to a complication in the sickness or disease of the patient. Further, this question is important since all healthcare providers would want to know if his or her patient has been hospitalized for any reason since the last contact with the patient. FIG. 7 illustrates a directive being given, in the form of a text message, to the patient via PCD 10 which has originated from the second intelligent database at the doctor's office. As will be discussed hereinafter, this directive may be formed automatically by an algorithm within the second intelligent database in response to the data collected by the second intelligent database or may be a diversion therefrom wherein the doctor manually overrides the algorithmically formed directive prior to sending the directive to the patient on her PCD 10.

With continuing reference to FIGS. 4 through 7, it is illustrated that a variety of “answering protocols” can be used. For instance, in FIG. 4, the patient is being asked to enter a number of which only the patient currently knows (i.e., her weight). Accordingly, when posed with this type of question, a numeric touch sensitive key pad is displayed so that the patient can enter the appropriate information. This type of illuminated key pad is also used to answer a question regarding the patient's blood pressure. As shown in FIG. 5, a range of numbers are illuminated to permit the patient to rate a symptom (in this case, whether there has been any swelling in the legs today). Depending on the question, the range of numbers can be three to ten or more. The example of FIG. 5 illustrates five numbers within a range. As shown in FIG. 6, a “yes” or “no” question is being asked. Accordingly, two choices are illuminated, namely “yes” and “no”. As shown in FIG. 7, a simple one choice acknowledgment is being sought (OK?) to confirm that the patient has received the instructions or directive transmitted by the doctor. As noted before, these examples are not complete, but merely illustrative of the types of questions displayed on the PCD and the potential answers sought thereafter. Other examples, but again not a complete list, of questions that may be asked relate to breathlessness, ankle swelling, fatigue, endurance, dizziness, urine output, temperature, general health and quality of sleep. A simple question might ask whether the patient wishes to be contacted today by the physician's office.

Referring to FIG. 8, a personal computer 18 is shown having a monitor 20 wherein it is illustrated that a drug intervention has been formed by an algorithm within the second intelligent database in response to data collected from the Server 14 sent thereto by the patient through the Internet 12 on her PCD 10. A question is posed to the healthcare provider whether the patient should be directed to take a certain medication due to changes in her health condition. In this example, the doctor is going to concur with the suggested directive prior to it being sent to the patient on her PCD 10.

To illustrate the use of the patient management system of the present invention, a single patient communicating with a single doctor will be described hereinbelow. of course, it is understood, that a single healthcare provider or doctor could have a plurality of patients with which to communicate. And as stated before, the system could be configured such that a single patient could use her PCD to communicate with a plurality of doctors.

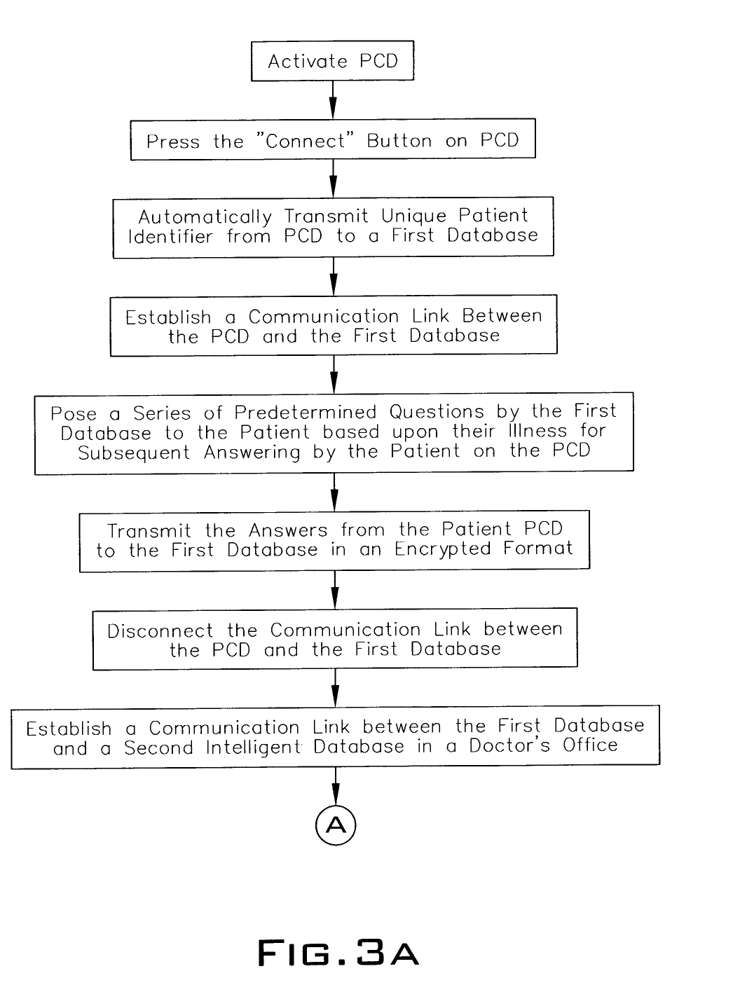

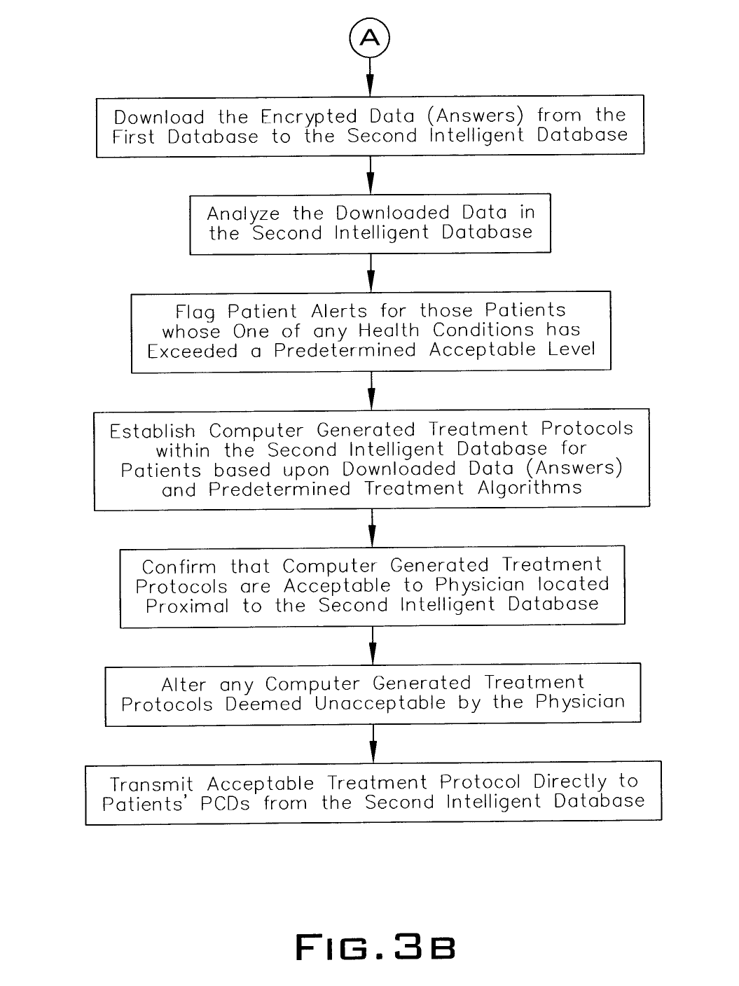

Referring to FIGS. 3A and 3B, a series of steps are shown which represent how a patient communicates her health condition (or health data) to a doctor and how such data is used to decide whether a drug or other intervention should be directed to the patient. First, the PCD must be activated. In the preferred embodiment, the PCD can be an “always on” unit which can be powered by a battery and recharged when necessary through an AC adapter. A communication link is next established with the PCD and the first intelligent database, or Server 14, through the Internet 12. This is done by engaging a “connect” button on PCD 10. Once the communication link is established, PCD 10 automatically sends the patient's unique ID number which has been previously assigned to the patient. Next, a series of predefined questions are posed to the patient by Server 14 (like those illustrated in FIGS. 4-6), or stored in the PCD 10 and posed by the PCD 10 to the patient. These questions are directed towards the patient based on their health condition. As such, they can be changed by someone having permissible access to Server 14 since some questions may no longer be relevant as the patient's health improves and likewise, additional questions may become necessary if the patient's health declines. It is also important to understand that the system of the present invention is not limited to the same series of predefined questions for all patients of a single doctor. Since each patient's health condition may vary, the questions posed to each patient on their PCD 10 may be tailored for each particular patient. The answers that are provided by the patient populate fields within the first database (Server 14). No patient identifying information is kept in the first database. Upon the patient answering all of the questions directed to the patient on her PCD 10 by Server 14, the answers are transmitted to Server 14 in an encrypted form where the data is stored. Thereafter, the link between PCD 10 and Server 14 is severed, unless the PCD 10 is in the “always on” mode. As noted above, a doctor can have a plurality of patients utilizing the patient management system of the present invention. Accordingly, all of a doctor's patients using the system can store their health data on the same Server 14. Continuous sampling can be used so patients can enter data at different times. However, it is understood that multiple servers could be employed if needed or desired.

At least once a day, in the preferred method, the doctor's office downloads the patient health data stored in Server 14. This is done by establishing a link between the second intelligent database (located in or in close proximity to the doctor's office) and Server 14. The second intelligent database stores the encrypted data collected from Server 14 and decrypts it for analysis. Once decrypted and analyzed, if a patient flag is generated, the health data for each flagged patient is subjected to a predefined algorithm which results in a computer generated directive for each flagged patient. These directives may include suggested drug protocols or other health related directives relating to treatment. The patient alert system embedded within the algorithms alerts the healthcare provider to a potential indication of a deterioration in the patient's condition in response to the encrypted collected downloaded data. For instance, the algorithm may have an embedded alert flag which notifies the healthcare provider that the patient has gained weight, indicating that the patient may be retaining fluid. Or, as another example, the algorithm may have an embedded alert flag which alerts the healthcare provider that the patient is significantly more short of breath today than yesterday. These alert flags (or symptom scores) can then be used to adjust medication or give the healthcare provider enough information to decide whether the doctor should intervene further (it is of course understood that the aforementioned examples are merely two alert examples from a plurality of alerts that the doctor may use to advise his or her patient). It is not likely that every patient under the care of a particular doctor utilizing the system will flag an alert for each and every patient. For instance, the doctor may have 300 patients under care for heart disease and heart failure transmitting data each day, but only twenty patients on a specific download trigger an alert. That is not to say however, that not every patient could trigger an alert on any given day. It is merely important to understand that the algorithms are attempting to “sort-out” symptomatic patients who may need care above what they are currently receiving on that particular day by placing a flag next to their name in response to the information provided by the patient. Nothing in the system prohibits the doctor or his staff from looking at the report of each and every patient that has used the system that day. Further, noting that a particular patient may not have responded over a few days period may alert the doctor or staff to check on the actual health status of that patient by direct phone contact.

Prior to transmitting the directive to the patient which has been formed by the algorithm in response to the collected data, the doctor has an opportunity to customize the directive by overriding the computer generated drug protocol or directive. As shown in FIG. 8, a personal computer 18 having a monitor 20 is shown wherein a healthcare provider is being asked to concur or not to concur with the computer generated suggested intervention protocol. If the doctor answers “yes”, then nothing further is required. However, if the doctor answers “no”, then a screen will be provided so that the doctor can change the suggested intervention protocol to a treatment which he feels is required. This information is then transmitted to the patient on her PCD 10 from the second intelligent database. It is very important to note that the patient management system of the present invention is not meant to replace the actual care typically provided by a doctor to his patient, but is one used in conjunction with the normal care provided by the doctor so that everyday health condition analysis can be performed, if necessary, for any given patient. This results in potentially reducing the number of hospitalizations that may occur with the changing of a patient's condition from one day to the next without the patient directly notifying her doctor (i.e., calling the doctor on the phone or going in for an office visit). Accordingly, the directives that are transmitted to the patient, such as an increase in a certain drug treatment, can potentially reduce such hospital visits in between actual doctor office visits and in some instances thwart a catastrophic health decline.

It is noted that nothing herein limits the use of the system of the present invention as merely a once-a-day reporting and interactive system. In fact, it could be used a plurality of times on any one given day. It is also noted that the system, and in particular PCD 10, can be used as a medication reminder or calendar. For those patients taking a multitude of different drugs, PCD 10 can be sent information or programmed to remind the patient that it is now time to take a particular medication. It is still further noted the system can also be used as a reminder service to notify the doctor that a particular patient is now stable at a specific drug dosage or that an optimal drug dosage has or has not been reached. Since it may be desirable to titrate a patient to a target medication dose to achieve certain clinical outcomes, the doctor can be notified that such has not been reached, thereby prompting the doctor to increase the drug dosage at his discretion by sending a text message to the patient on her PCD 10.

A third database can be provided in the patient management system of the present invention for use in educating all patients within the system. In the preferred embodiment, wherein the third database is employed, such third database is connected to the Internet 12. The third database is used to help patients with health care and patient education. Most patients that suffer from heart disease, for example, know very little about the disease itself and the symptoms that may be indicative that the disease process is getting out of control. It is well known that therapeutic lifestyle changes can play a very important role in the recovery from almost all chronic illnesses and helps in reducing reoccurring undesirable events. A patient could use the third database to learn what should be the typical sodium intake amount for a person of her health condition and strategies for staying within her sodium budget.

Group messaging from a third database leads the patients through a series of educational modules, thereby empowering the patients to better understand their respective health condition thereby encouraging them to take a more proactive management of their daily health condition. In an alternate embodiment of the system using the third database, education modules can be flashed onto PROMs in the communication devices in the possession of the patient so that a library of information is readably available to the patient for sequential or repetitive viewing and for further reference in the future. The PROMs can of course be simply updated through connection to an update service via the Internet 12 or by direct connection with an update center (i.e., the doctor's office). The third database can also be used as a benchmark tool to measure the efficacy of the entire patient management system. It can be used to gather information on benchmarks of patient progress (i.e., serial scores on quality of life questionnaires since the chronic illness occurred, measurements of exercise tolerance, certain organ function and other relevant information). This gathered information then can be used to rate the effectiveness of the patient management system and if the provider of the system is reaching the goals set forth in the use of the system. The third database can also be used to track the number of patient alerts generated, the number of interventions delivered and what percentage of the interventions generated by the algorithms were delivered as compared to those that were customized by the physician.

Still further alternate embodiments of the system include the ability to a keep a complete patient electronic record history that would be available to a healthcare provider upon permission being given by the patient. Accordingly, a complete patient history from one physician could be transferred or made available to another physician almost instantaneously. This use has clear benefits wherein a family doctor could easily provide the entire health history to a specialist, such as a cardiologist, simply by permitting access to the specialist to the second database of the family doctor containing the health history. This could also be used to minimize mistakes in adverse drug prescription interactions, whereby one physician could easily check to see exactly what medications a patient is currently taking which may have been previously prescribed by another physician but not brought to his attention.

Yet still further alternate embodiments of the patient management system of the present invention includes a secure billing module for use by the patient, doctor and insurance company. Upon systematic updates of the third database with inclusion of the patient's full medical history, the secure billing module can be used to track exactly what care has been provided to date and how much that care has cost. Further, it can state whether the insurance company or patient is responsible for payment of the health care services provided and whether the physician has been paid for such services.

Equivalent steps can be substituted for the ones set forth above such that they perform the same method in the same way for achieving the same result.

Having thus described the invention, what is claimed and desired to be secured by letters patent is:

1. A method for remotely monitoring and managing a patient's health condition by a healthcare provider, the steps of the method comprising:
a) providing a personal communication device to the patient, the personal communication device capable of communicating with a global information computer network;
b) providing a first intelligent database residing on the global information computer network;
c) directing the patient to activate the personal communication device each day;
d) establishing a communication link between the personal communication device and the first intelligent database through the global information computer network;
e) permitting the first intelligent database to pose a series of questions to the patient through the global information computer network such that the questions appear on the personal communication device;
f) directing the patient to provide health condition data in response to questions posed by the first intelligent database by engaging keys on the patient's personal communication device;
g) transmitting the health condition data in an encrypted form from the personal communication device to the first intelligent database through the global information computer network;
h) providing a second intelligent database located proximal to the healthcare provider;
i) establishing a communication link between the second intelligent database and the first intelligent database;
j) downloading the patient's encrypted health condition data from the first intelligent database to the second intelligent database;
k) analyzing the patient's health condition data by the second intelligent database,
l) providing a patient alert system within the second intelligent database so that upon analyzing the patient's health condition data, a changing health condition can trigger an alert;
m) providing a plurality of treatment algorithms and drug protocols in the second intelligent database which are capable of responding to a patient alert;
n) forming a treatment directive in response to the treatment algorithms and drug protocols being applied to the patient alert; and
o) transmitting the treatment directive to the patient's personal communication device.

2. The method of claim 1, wherein the personal communication device communicates with the global information computer network through a wireless access protocol.

3. The method of claim 2, wherein the personal communication device is chosen from the group consisting of cellular phones, patient communication devices, pagers, satellite receivers and text messaging devices.

4. The method of claim 1, wherein the personal communication device communicates with the global information computer network through a land line access protocol.

5. The method of claim 4, wherein the land line access protocol is chosen from the group consisting of cable modems, DSL lines, dial-up telephone modems and fiber optic cable devices.

6. The method of claim 4, wherein the personal communication device is a personal computer.

7. The method of claim 1, wherein the step of establishing a communication link between the personal communication device and the first intelligent database through the global information computer network comprises the steps of:
a) engaging a connection button on the personal communication device; and
b) transmitting automatically a unique patient identification number.

8. The method of claim 7, wherein the unique patient identification number is an encrypted string of numbers.

9. The method of claim 1, wherein the communication link established between the first and second databases is effected by a land line access protocol.

10. The method of claim 1, wherein the communication link established between the first and second databases is effected by a wireless access protocol.

11. The method of claim 1, wherein the patient alert system triggers the alert upon analyzing the patient's health condition data and finding that a certain numeric figure representing a threshold has been exceeded either below or above a predefined acceptable level.

12. The method of claim 1, wherein the step of forming a treatment directive in response to the treatment algorithms and drug protocols being applied to the patient alerts comprises the steps of:
a) permitting the second intelligent database to propose a suggested treatment directive for delivery to the patient; and
b) accepting or overriding the proposed suggested treatment directive.

13. The method of claim 1, wherein the treatment directive transmitted to the patient's personal communication device appears as a text message on a display screen.

14. The method of claim 13, wherein the text message contains instructions for the patient to take at least one medication at a particular dosage level.

15. The method of claim 1, wherein the step of transmitting the treatment directive to the patient's personal communication device is effected by establishing a direct communication link between the second intelligent database and the personal communication device.

16. The method of claim 1, further comprising the step of:
a) providing a third database for the purpose of retrieving information regarding a certain illness.

17. The method of claim 1, wherein the personal communication device is programmed with a library of information regarding a certain illness for use by the patient as a reference source of the information.

18. The method of claim 1, wherein the global information computer network is the Internet.

19. A method for remotely monitoring and managing a patient's health condition by a healthcare provider, the steps of the method comprising:
a) providing a wireless personal communication device to the patient, the wireless personal communication device capable of communicating with a global information computer network;
b) providing a first intelligent database residing on the global information computer network;
c) directing the patient to activate the wireless personal communication device periodically;
d) establishing a communication link between the wireless personal communication device and the first intelligent database through the global information computer network;
e) permitting the first intelligent database to pose a series of questions to the patient through the global information computer network such that the questions appear on the wireless personal communication device;
f) directing the patient to provide health condition data in response to questions posed by the first intelligent database by engaging keys on the patient's wireless personal communication device;
g) transmitting the health condition data in an encrypted form from the wireless personal communication device to the first intelligent database through the global information computer network;
h) providing a second intelligent database located proximal to the healthcare provider;
i) establishing a communication link between the second intelligent database and the first intelligent database;
j) downloading the patient's encrypted health condition data from the first intelligent database to the second intelligent database;
k) analyzing the patient's health condition data by the second intelligent database,
l) providing a patient alert system within the second intelligent database so that upon analyzing the patient's health condition data, a changing health condition can trigger an alert;
m) providing a plurality of treatment algorithms and drug protocols in the second intelligent database which are capable of responding to a patient alert;
n) permitting the second intelligent database to form a proposed treatment directive in response to the treatment algorithms and drug protocols being applied to the patient alert;
o) reviewing the proposed treatment directive;
p) forming an actual treatment protocol by accepting or overriding the proposed treatment directive; and
q) transmitting the actual treatment directive to the patient's personal communication device.

20. The method of claim 19, wherein the wireless personal communication device is chosen from the group consisting of cellular phones, patient communication devices, pagers, satellite receivers and text messaging devices.

21. The method of claim 19, wherein the step of establishing a communication link between the wireless personal communication device and the first intelligent database through the global information computer network comprises the steps of:
a) engaging a connection button on the wireless personal communication device; and
b) transmitting automatically a unique patient identification number.

22. The method of claim 21, wherein the unique patient identification number is an encrypted string of numbers.

23. The method of claim 19, further comprising the step of
a) providing a third database for the purpose of retrieving information regarding a certain illness.

24. The method of claim 19, wherein the patient alert system triggers the alert upon analyzing the patient's health condition data and finding that a certain numeric figure representing a threshold has been exceeded either below or above a predefined acceptable level.

25. A method for remotely monitoring and managing a patient's health condition by a healthcare provider, the steps of the method comprising:
a) providing a personal communication device to the patient, the personal communication device capable of communicating with a global information computer network;
b) providing a first intelligent database residing on the global information computer network;
c) directing the patient to activate the personal communication device so that is it is always on;
d) establishing a communication link between the personal communication device and the first intelligent database through the global information computer network;
e) posing a series of questions to the patient on a screen of the personal communication device;
f) directing the patient to provide health condition data in response to questions posed on the patient's personal communication device by engaging keys;
g) transmitting the health condition data in an encrypted form from the personal communication device to the first intelligent database through the global information computer network;
h) providing a second intelligent database located proximal to the healthcare provider;
i) establishing a communication link between the second intelligent database and the first intelligent database;
j) downloading the patient's encrypted health condition data from the first intelligent database to the second intelligent database;
k) analyzing the patient's health condition data by the second intelligent database,
l) providing a patient alert system within the second intelligent database so that upon analyzing the patient's health condition data, a changing health condition can trigger an alert;
m) providing a plurality of treatment algorithms and drug protocols in the second intelligent database which are capable of responding to a patient alert;
n) forming a treatment directive in response to the treatment algorithms and drug protocols being applied to the patient alert; and
o) transmitting the treatment directive to the patient's personal communication device.
